(19) United States
(12) Patent Application Publication
(10) Pub. No.: US 2023/0374552 A1
(43) Pub. Date: Nov. 23, 2023

(54) METHOD OF PRODUCING HYDROGEN PEROXIDE USING MICROORGANISM OF THE GENUS DESEMZIA

(71) Applicant: Gwangju Institute of Science and Technology, Gwangju (KR)

(72) Inventors: Hor Gil Her, Gwangju (KR); Yong Seok Ko, Gwangju (KR); You Ri Yang, Gwangju (KR); Sunil Subhash Ghatge, Gwangju (KR)

(21) Appl. No.: 18/165,620

(22) Filed: Feb. 7, 2023

(30) Foreign Application Priority Data
May 23, 2022 (KR) 10-2022-0062765

(51) Int. Cl. C12P 3/00; C12N 1/20; B09C 1/08

(52) U.S. Cl. CPC C12P 3/00; C12N 1/20; B09C 1/08

(57) ABSTRACT

The present disclosure relates to a method of producing hydrogen peroxide (H₂O₂) using a microorganism of the genus Desemzia. According to the present disclosure, to overcome transportation cost problems associated with a special transport container, a low temperature and a stabilizer, other than the production cost of hydrogen peroxide (H₂O₂), Desemzia sp. strain C1, a hydrogen peroxide (H₂O₂)-producing microorganism, is isolated, cultured, and used to produce hydrogen peroxide (H₂O₂), and thus it may overcome the transportation cost problems and may be effectively used in an environmentally friendly and economical way in pollutant degradation processes, including sewage treatment plants.

### CROSS-REFERENCE TO RELATED APPLICATIONS

This application claims the benefit of priority of Korean Patent Application No. 10-2022-0062765, filed May 23, 2022, which is hereby incorporated herein by reference in its entirety.

### REFERENCE TO AN ELECTRONIC SEQUENCE LISTING

The instant application contains a Sequence Listing which has been submitted electronically and is hereby incorporated by reference in its entirety. The Sequence Listing was created on Dec. 16, 2022, is named “POPB224112US-SequenceListing.xml” and is 7 kilobytes in size.

### BACKGROUND

### 1. Technical Field

The present disclosure relates to Desemzia sp. strain C1 producing large amounts of hydrogen peroxide, and a method of producing hydrogen peroxide using the same.

### 2. Related Art

Hydrogen peroxide is an environmentally friendly oxidizing agent that is widely applied in chemical and environmental processes. The production of hydrogen peroxide (H₂O₂) by microorganisms has been reported mainly in lactic acid bacteria such as Streptococcus, Lactobacillus and Pediococcus. Lactic acid-producing bacteria known to produce hydrogen peroxide (H₂O₂) exist as normal flora in mammals and can cause infectious diseases while being ecologically closely related to mammals. The genus Lactobacillus is mainly found in mammalian vaginas and dairy products, and the genus Streptococcus is a pathogen that causes pneumonia, necrotizing fasciitis, and bacteremia. Hydrogen peroxide (H₂O₂)-producing bacteria produce hydrogen peroxide (H₂O₂) using the enzymes lactate oxidase, pyruvate oxidase, oxalate oxidase and NADH oxidase involved in basic carbon source or energy metabolisms, lack catalase, peroxidase and peroxidase-regulating enzymes (OxyR, PerR), which are enzymes degrading hydrogen peroxide (H₂O₂), and thus hydrogen peroxide (H₂O₂) produced by these bacteria accumulates around the cells. Hydrogen peroxide (H₂O₂) is an environmentally friendly oxidizing agent that generates little or no pollutants, and is used as a bleaching agent for paper pulp, a final oxidizing agent for propene epoxide, and a disinfectant. Fenton reaction, ozone treatment, UV treatment and the like result in the generation of hydroxyl radicals (—OH) having a very high oxidization potential from hydrogen peroxide (H₂O₂). —OH radicals have the highest oxidation potential (+2.8 V) among reactive oxygen species, which can be generated from oxygen, and degrade compounds by increasing their reactivity through non-specific quick reactions (10⁸ to 10¹⁰ M⁻¹s⁻¹).

Over 95% of hydrogen peroxide (H₂O₂) produced worldwide is produced by the anthraquinone oxidation process (AOP). AOP is known as an environmentally friendly and efficient synthesis method because it synthesizes hydrogen peroxide (H₂O₂) from oxygen (O₂) and hydrogen (H₂) gases at a relatively low pressure of 4 to 5 atm and a relatively low temperature of 40 to 50° C. and emits only oxygen (O₂) and hydrogen peroxide (H₂O₂) as by-products. However, since hydrogen peroxide (H₂O₂) reacts with trace metal ions due to its high reactivity and is likely to be decomposed under natural conditions, a special transport container, a low temperature of 10° C. or less and a stabilizer for stabilizing hydrogen peroxide (H₂O₂) are required. This increases the price of hydrogen peroxide (H₂O₂), and indeed, the production cost of hydrogen peroxide (H₂O₂) produced using AOP is 100 $/ton, whereas the storage and transportation cost of hydrogen peroxide is 300 $/ton.

Recently, there have been reported studies on the degradation of environmental pollutants by the Bio-Fenton reaction using microorganisms and hydrogen peroxide (H₂O₂) generated from microorganism-derived enzymes. Hydrogen peroxide (H₂O₂) production using microorganisms is expected to be an environmentally friendly and economical method in processes of degrading pollutants, including sewage treatment plants, because hydrogen peroxide (H₂O₂) can be produced using a simple carbon source in the environment and degradation metabolites can be incorporated into microbial metabolism naturally. Desemzia sp. strain C1 isolated from a waste oil-contaminated environment according to the present disclosure is an environment-derived microorganism having no known pathogenicity while producing large amounts of hydrogen peroxide (H₂O₂). Thus, it is expected that Desemzia sp. strain C1 may be applied to an in situ hydrogen peroxide (H₂O₂) production process and a method of degrading environmental organic pollutants by the Bio-Fenton reaction using hydrogen peroxide.

### SUMMARY

The inventors of the present disclosure have made extensive research efforts to overcome transportation cost problems associated with a special transport container, a low temperature and a stabilizer, other than the production cost of hydrogen peroxide (H₂O₂). As a result, the present inventors have found that, when Desemzia sp. strain C1, a hydrogen peroxide (H₂O₂)-producing microorganism, is isolated from waste oil-contaminated soil and cultured, it may overcome the problem associated with the transportation cost of hydrogen peroxide, and may be used as a hydrogen peroxide (H₂O₂)-producing microorganism in an environmentally friendly and economical way in pollutant degradation processes, including sewage treatment plants.

Therefore, an object of the present disclosure is to provide a method of producing hydrogen peroxide (H₂O₂) using a microorganism of the genus Desemzia.

Other objects and advantages of the present disclosure will become more apparent from the following detailed description of the disclosure, the appended claims and the accompanying drawings.

However, objects to be achieved by the present disclosure are not limited to the object mentioned above, and other objects not mentioned herein will be clearly understood by those of ordinary skill in the art from the following description.

Hereinafter, various embodiments described herein will be described with reference to figures. In the following description, numerous specific details are set forth, such as specific configurations, compositions, and processes, etc., in order to provide a thorough understanding of the present disclosure. However, certain embodiments may be practiced without one or more of these specific details, or in combination with other known methods and configurations. In other instances, known processes and preparation techniques have not been described in particular detail in order to not unnecessarily obscure the present disclosure. Reference throughout this specification to “one embodiment” or “an embodiment” means that a particular feature, configuration, composition, or characteristic described in connection with the embodiment is included in at least one embodiment of the present disclosure. Thus, the appearances of the phrase “in one embodiment” or “an embodiment” in various places throughout this specification are not necessarily referring to the same embodiment of the present disclosure. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present disclosure pertains.

Throughout the present specification, it is to be understood that when any part is referred to as “comprising” any component, it does not exclude other components, but may further comprise other components, unless otherwise specified.

According to one aspect of the present disclosure, the present disclosure provides a method of producing hydrogen peroxide (H₂O₂) using a microorganism of the genus Desemzia.

The initiators of the present disclosure have made extensive research efforts to overcome the enormous transportation cost problems associated with a special transport container, low-temperature maintenance and the use of a stabilizer, other than the production cost of hydrogen peroxide (H₂O₂). As a result, the present inventors have found that, when Desemzia sp. strain C1 (accession number: KCTC15203BP), a hydrogen peroxide (H₂O₂)-producing microorganism, is isolated from waste oil-contaminated soil and cultured, it may overcome the above-described problems.

As used herein, the term “microorganism” refers to small organisms invisible to the naked eye, which are generally single-cellular organisms or even multicellular organisms, which have not undergo morphological and functional cell differentiation and can live independently as single cells. Microorganisms include eukaryotes and eubacteria, which comprise cells having a nucleus surrounded by a nuclear membrane, such as protozoa, algae and fungi, prokaryota, which comprise cells with indistinguishable nuclei due to lack of a nuclear membrane, such as archaebacterial, and viruses and viroids that have genetic material but are difficult to see as cells because there is no translation device and no metabolic function. Microorganisms generally include fungi, protozoa, bacteria, viruses, algae, and the like.

As used herein, the term “hydrogen peroxide (H₂O₂)” refers to an inorganic compound in which one oxygen atom is attached to a water (H₂O₂) molecule. Hydrogen peroxide is a transparent compound composed of oxygen and hydrogen, and appears almost transparent in aqueous solution. It is the simplest and most commonly present peroxide. It has weak acidity, is usually difficult to exist at high concentration, is diluted in water to make a hydrogen peroxide solution, and is also used as a disinfectant.

As used herein, the term “Desemzia” refers to a bacteria genus belonging to the family Carnobacteriaceae.

As used herein, the term “Streptococcus” refers to a generic term for Gram-positive bacteria belonging to the genus Streptococcus of the family Streptococcaceae of the order Lactobacilliales. Streptococcus was named because the colony grows like a chain due to cell division occurring along one axis, in contrast to Staphylococcus aureus.

As used herein, the term “Lactobacillus” refers to lactic acid bacteria, and the term “lactic acid bacterial species” generally refer to probiotics (good bacteria) found in the human digestive tract and urinary tract. Lactobacillus may be consumed for anti-diarrhea and intestinal health, allows the body to break down food and absorb nutrients, and can fight pathogenic organisms that can cause disease.

As used herein, the term “Pediococcus” refers to a genus of Gram-positive lactic acid bacteria belonging to the family Lactobacillaceae. They usually occur in pairs or tetrads, and divide along two planes of symmetry, as do the other lactic acid cocci genera Aerococcus and Tetragenococcus.

As used herein, the term “Bifidobacterium” refers to a genus of Gram-positive, non-motile, often branched anaerobic bacteria. B. dentium strains were isolated from the vagina and mouth of mammals, including humans, but are widely distributed in the gastrointestinal tract. Bifidobacteria are one of the major genera of bacteria that make up the mammalian gut flora, and some bifidobacteria are also used as probiotics.

According to a specific embodiment of the present disclosure, the microorganism is a microorganism having a 16S rDNA represented by SEQ ID NO: 1.

According to the present disclosure, the 16S rDNA nucleotide sequence of the microorganism is 1,408 bp in total length. As a result of analyzing the 16S rDNA nucleotide sequence of the microorganism comparatively with those of conventionally known standard strains in NCBI BLAST, it could be confirmed that the 16S rDNA nucleotide sequence of the microorganism was 99.29%, 96.40%, and 95.51% identical to those of Desemzia inserta, Pisciglobus halotoerans, and Carnobacterium viridans, respectively.

According to a specific embodiment of the present disclosure, the microorganism is a microorganism comprising the nucleic acid represented by SEQ ID NO: 1, or a Desemzia sp. strain C1 (accession number KCTC15203BP).

According to a specific embodiment of the present disclosure, the microorganism uses lactate oxidase.

As used herein, the term “enzyme” refers to a biocatalyst that binds to a substrate to form an enzyme-substrate complex, thereby lowering the activation energy of a chemical reaction, thereby increasing the rate of metabolism. In some cases, enzymes perform a bioprotective function of regulating the rate and convert substrates into other molecules known as products. Almost all metabolic processes in cells require enzymatic catalysis because they must occur at a rate fast enough to sustain life. Metabolic pathways rely on enzymes to catalyze individual steps. Enzymes are known to catalyze more than 5,000 types of biochemical reactions.

As used herein, the term “lactate oxidase” is also called lactate 2-monooxygenase, and refers to an enzyme that catalyzes a chemical reaction. This enzyme belongs to the family of oxidoreductases, specifically those acting on single donors with O₂ as oxidant and incorporation of two atoms of oxygen into the substrate (oxygenase). Other names in common use include lactate oxidative decarboxylase, lactate oxidase, lactic oxygenase, lactate oxygenase, lactic oxidase, L-lactate monooxygenase, lactate monooxygenase, and L-lactate-2-monooxygenase. This enzyme participates in pyruvate metabolism.

As used herein, the term “pyruvate oxidase” refers to an enzyme that catalyzes a chemical reaction. This enzyme belongs to the family of oxidoreductases, specifically those acting on the aldehyde or oxo group of donor using oxygen as an acceptor. Other names in common use include pyruvic oxidase and phosphate-dependent pyruvate oxidase. This enzyme participates in pyruvate metabolism.

As used herein, the term “oxalate oxidase” refers to an enzyme that catalyzes a chemical reaction. This enzyme belongs to the family of oxidoreductases, specifically those acting on the aldehyde or oxo group of donor using oxygen as an acceptor. Other names in common use include aero-oxalo dehydrogenase and oxalic acid oxidase. This enzyme participates in glyoxylate and dicarboxylate metabolism, and uses manganese as a cofactor.

As used herein, the term “NADH oxidase” refers to a membrane-bound enzyme complex that faces the extracellular space. It can be found in the plasma membrane as well as in the membranes of phagosomes used by neutrophil white blood cells to engulf microorganisms. Human isoforms of the catalytic component of the complex include NOX1, NOX2, NOX3, NOX4, NOX5, DUOX1, and DUOX2.

As used herein, the term “catalase” refers to a common enzyme found in nearly all living organisms exposed to oxygen (such as bacteria, plants, and animals) which catalyzes the decomposition of hydrogen peroxide to water and oxygen. Catalase is a very important enzyme that protects cells from oxidative damage caused by reactive oxygen species (ROS), and has one of the highest turnover numbers of all enzymes. One catalase molecule can convert millions of hydrogen peroxide molecules into water and oxygen per second.

As used herein, the term “peroxidase” refers to an enzyme that catalyzes the reaction AH₂+H₂O₂→A+2H₂O using hydrogen peroxide (H₂O₂) in the dehydrogenation of a substrate. Peroxidase is widely present in plant tissues, but is present in animal tissues in small amounts, and particularly, is contained in horseradish in large amounts. It is a complex protein having a molecular weight of 44,000 and containing one protohematin (trivalent iron attached to protoporphyrin) per molecule, and is an iron porphyrin protein, like catalase. When the protein portion and protohematin are separated from each other, the enzymatic activity is lost, but when the two are combined, the enzymatic activity returns to the original state.

According to a specific embodiment of the present disclosure, the microorganism is nonpathogenic.

As used herein, the term “nonpathogenic” refers to a microorganism that has no ability to cause disease.

According to a specific embodiment of the present disclosure, the microorganism belongs to the same clade as Desemzia inserta.

As used herein, the term “clade” refers to a group of organisms belonging to a single lineage (composed of a common ancestor and all of its descendants) in a phylogenetic tree.

According to a specific embodiment of the present disclosure, the microorganism belongs to a phylogenetic branch distinct from Desemzia inserta.

According to the present disclosure, a H₂O₂-producing microorganism was isolated from waste oil-contaminated soil, and it was identified to belong to the genus Desemzia and named Desemzia sp. strain C1. Since the genus Desemzia was reported by Steinhaus in 1941, cases of human and mammalian infections with the genus Desemzia have not been reported, and thus the genus Desemzia is considered a non-pathogenic bacterium. Desemzia sp. strain C1 produced 0.23 mM hydrogen peroxide (H₂O₂) in BHI broth, which was four times larger than that of S. oralis, a well-known strain producing hydrogen peroxide (H₂O₂). In a resting-cell assay using 10 mM lactate as a substrate (initial cell concentration O.D.₆₀₀=2.5), Desemzia sp. strain C1 produced 0.75 mM hydrogen peroxide (H₂O₂), which was about three times higher than that of S. oralis. That is, in the resting-cell assay using lactate as a substrate, Desemzia sp. strain C1 could produce hydrogen peroxide (H₂O₂) in an amount 3.5 times larger than that of a method of producing hydrogen peroxide (H₂O₂) using a nutrient broth. Desemzia sp. strain C1 is not pathogenic and has excellent hydrogen peroxide (H₂O₂) production ability, and thus it is expected that Desemzia sp. strain C1 may be applied to an in situ hydrogen peroxide (H₂O₂) production process and a method of degrading organic pollutants in the environment by the Bio-Fenton reaction using hydrogen peroxide (H₂O₂).

As used herein, the term “Prussian blue” refers to a dark blue pigment produced by oxidation of ferrous ferricyanide salts. Prussian blue is also kali blue because the composition is KFe[Fe(CN)₆], and is also called kali blue, and ammonium iron(III) hexacyanoferrate(II) [(NH₄)Fe[Fe(CN)₆] is also called soda blue. Prussian blue was the first modern synthetic pigment, and is prepared as a very fine colloidal dispersion, because the compound is not soluble in water. It contains variable amounts of other ions and its appearance depends sensitively on the size of the colloidal particles. In medicine, orally administered Prussian blue is used as an antidote for certain kinds of heavy metal poisoning, e.g., by thallium(I) and radioactive isotopes of cesium. This therapy exploits the compound's ion-exchange properties and high affinity for certain “soft” metal cations.

As used herein, the term “ferricyanide” refers to the anion [Fe(CN)₆]³⁻, and is also called hexacyanoferrate(III) or hexacyanidoferrate(III). The most common salt of this anion is potassium ferricyanide, a red crystalline material that is used as an oxidant in organic chemistry.

According to another aspect of the present disclosure, the present disclosure provides a composition for degrading pollutants comprising a hydrogen peroxide produced by the method of producing hydrogen peroxide (H₂O₂) using a microorganism of the genus Desemzia.

According to a specific embodiment of the present disclosure, the pollutants are organic pollutants.

According to another aspect of the present disclosure, the present disclosure provides a method for degrading one or more pollutants, the method comprising producing hydrogen peroxide by a method as described herein, and contacting the one or more pollutants with the hydrogen peroxide under conditions sufficient to degrade the pollutant. Each pollutant can be, for example, an organic pollutant.

According to another aspect of the present disclosure, the present disclosure provides a composition for degrading pollutants comprising a hydrogen peroxide produced by a microorganism comprising the nucleic acid represented by SEQ ID NO: 1, or a Desemzia sp. strain C1 (accession number KCTC15203BP).

According to a specific embodiment of the present disclosure, the microorganism uses lactate oxidase.

According to another aspect of the present disclosure, the present disclosure provides a method for degrading one or more pollutants, the method comprising producing hydrogen peroxide using a microorganism comprising the nucleic acid represented by SEQ ID NO: 1, or a Desemzia sp. strain C1 (accession number KCTC15203BP), and contacting the one or more pollutants with the hydrogen peroxide under conditions sufficient to degrade the pollutant. The pollutant can be, for example, an organic pollutant.

According to another aspect of the present disclosure, the present disclosure provides a composition for producing hydrogen peroxide comprising a microorganism of the genus Desemzia.

According to another aspect of the present disclosure, the present disclosure provides a composition for producing hydrogen peroxide comprising a microorganism comprising the nucleic acid represented by SEQ ID NO: 1, or a Desemzia sp. strain C1 (accession number KCTC15203BP).

According to another aspect of the present disclosure, the present disclosure provides a Desemzia sp. strain C1 (accession number KCTC15203BP).

The features and advantages of the present disclosure are summarized as follows:
(a) The present disclosure provides a method of producing hydrogen peroxide (H₂O₂) using a microorganism of the genus Desemzia.
(b) According to the present disclosure, to overcome transportation cost problems associated with a special transport container, a low temperature and a stabilizer, other than the production cost of hydrogen peroxide (H₂O₂), Desemzia sp. strain C1, a hydrogen peroxide (H₂O₂)-producing microorganism, is isolated, cultured, and used to produce hydrogen peroxide (H₂O₂), and thus it may overcome the transportation cost problems and may be effectively used in an environmentally friendly and economical way in pollutant degradation processes, including sewage treatment plants.

### BRIEF DESCRIPTION OF THE DRAWINGS

FIG. 1 shows Desemzia sp. strain C1 cultured in Prussian blue agar medium according to an example of the present disclosure.

FIG. 2 shows the results of analyzing the phylogenetic tree of Desemzia sp. strain C1 by molecular biological identification according to an experimental example of the present disclosure.

FIG. 3A shows growth curves of Desemzia sp. strain C1 and S. oralis, obtained according to an experimental example of the present disclosure. Strain C1 cultured in BHI (●) and TSBY (○), and S. oralis cultured in BHI (▾) and TSBY (Δ) are shown.

FIG. 3B shows the production of hydrogen peroxide (H₂O₂) by each of Desemzia sp. strain C1 and S. oralis according to an experimental example of the present disclosure. Strain C1 cultured in BHI (●) and TSBY (○), and S. oralis cultured in BHI (▾) and TSBY (Δ) are shown.

FIG. 4 shows the results of measuring the production of hydrogen peroxide (H₂O₂) by Desemzia sp. strain C1 depending on a substrate according to an experimental example of the present disclosure.

FIG. 5 shows the results of measuring the production of hydrogen peroxide (H₂O₂) by Desemzia sp. strain C1 depending on cell concentration according to an experimental example of the present disclosure. O.D.₆₀₀=0.5 (●), 1 (○), 2.5 (▾) and 5 (Δ).

FIG. 6A shows the production of hydrogen peroxide (H₂O₂) using lactate as a substrate according to an experimental example of the present disclosure. Strain C1 (●) and S. oralis (○).

FIG. 6B shows changes in lactate concentration during the production of hydrogen peroxide (H₂O₂) using lactate as a substrate according to an experimental example of the present disclosure. Strain C1 (●) and S. oralis (○).

### DETAILED DESCRIPTION

Hereinafter, the present disclosure will be described in more detail with reference to examples. These examples are only for illustrating the present disclosure in more detail, and it will be apparent to those of ordinary skill in the art that the scope of the present disclosure according to the subject matter of the present disclosure is not limited by these examples.

### EXAMPLES

### [Example 1] Isolation and Culture of Desemzia Sp. Strain C1

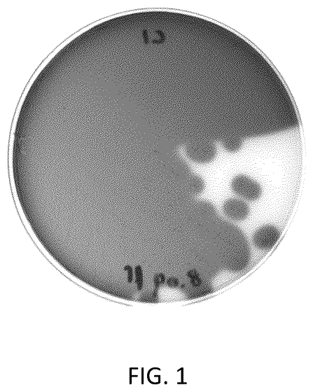

Soil exposed to waste oil for a long time was collected from an auto repair shop in Buk-gu, Gwangju, South Korea. Next, Desemzia sp. strain C1 (accession number: KCTC15203BP) was isolated based on the Prussian blue-zone formed by the reaction of ferric cyanide and hydrogen peroxide (H₂O₂) in Prussian blue agar medium. It was confirmed that Desemzia sp. strain C1 formed a large Prussian blue-zone by producing a large amount of hydrogen peroxide (H₂O₂) (FIG. 1).

### [Example 2] Molecular Biological Identification of Desemzia Sp. Strain C1

The 16S rDNA nucleotide sequence of Desemzia sp. strain C1 isolated according to the present disclosure was 1,408 bp in total length (Table 1, SEQ ID NO: 1). As a result of analyzing the 16S rDNA nucleotide sequence comparatively with those of conventionally known standard strains in NCBI BLAST, it was confirmed that the 16S rDNA nucleotide sequence of Desemzia sp. strain C1 was 99.29%, 96.40%, and 95.51% identical to Desemzia inserta, Pisciglobus halotoerans, and Carnobacterium viridans, respectively.

TABLE 1
16S rDNA nucleotide sequence of
Desemzia sp. strain C1

CGCTTCCTACCGGTGCTTGCACCGGGAAAGAAGAGTGGCG
GACGGGTGAGTAACACGTGGGTAACCTACCTACAAGCGGG
GGATAACATTCGGAAACGGATGCTAATACCGCATAGATCT
TTTTGTCTCCTGGCAGAAAGAAGAAAGACGGTTTCGGCTG
TCACTTGTAGATGGACCCGCGGCGCATTAGTTAGTTGGTG
AGGTAATGGCTCACCAAGACGATGATGCGTAGCCGACCTG
AGAGGGTGATCGGCCACACTGGGACTGAGACACGGCCCAG
ACTCCTACGGGAGGCAGCAGTAGGGAATCTTCCGCAATGG
ACGAAAGTCTGACGGAGCAACGCCGCGTGAGTGAAGAAGG
TTTTCGGATCGTAAAACTCTGTTGTTAGAGAAGAACAAGG
ATGAGAGTAACTGCTCGTCCCTTGACGGTATCTAACCAGA
AAGCCATGGCTAACTACGTGCCAGCAGCCGCGGTAATACG
TAGATGGCAAGCGTTGTCCGGATTTATTGGGCGTAAAGCG
AGCGCAGGCGGTTCTTTAAGTCTGATGTGAAAGCCCCTGG
CTCAACCAGGGGAGGGTCATTGGAAACTGGAGAACTTGAG
TGCAGAAGAAGAAAGTGGAATTCCATGTGGTAGCGGTGAA
ATGCGTAGATATATGGAGGAACACCAGTGGCGAAGGCGAC
TTTCTGGTCTGTAACTGACGCTGAGGCTCGAAAGCATGGG
GAGCAAACAGGATTAGATACCTTGGTAGTCCATGCCGTAA
ACGATGAGTGTTAAGTGTGGGAGGGTTTCCGCCTTTCAGT
GCTGCAGTTAACGCATTAAGCACTCCGCCTGGGGAGTACG
ACCGCAAGGTTGAAACTCAAAGGAATTGACGGGGGCCCGC
ACAAGCGGTGGAGCATGTGGTTTAATTCGAAGCAACGCGA
AGAACCTTACCAGGTCTTGACATCCTTTGACCACTCTAGA
GATAGAGCTTTCCCTTCGGGGACAAAGTGACAGGTGGTGC
ATGGTTGTCGTCAGCTCGTGTCGTGAGATGTTGGGTTAAG
TCCCGCAACGAGCGCAACCCTTATTATTAGTTGCCAGCAT
TCAGTTGGGCACTCTAGTGAGACTGCCGGTGATAAACCGG
AGGAAGGTGGGGATGACGTCAAATCATCATGCCCCTTATG
ACCTGGGCTACACACGTGCTACAATGGATGGTACAACGAG
TCGCAAAACCGCGAGGTTAAGCTAATCTCTTAAAGCCATT
CTCAGTTCGGATTGTAGGCTGCAATTCGCCTACATGAAGC
CGGAATCGCTAGTAATCGCGGATCAGAACGCCGCGGTGAA
TACGTTCCCGGGCCTTGTACACACCGCCCGTCACACCACG
AGAGTTTGTAACACCCAAGTCGGTGAGGTAACCTTCGGGA
GCCAGCCG

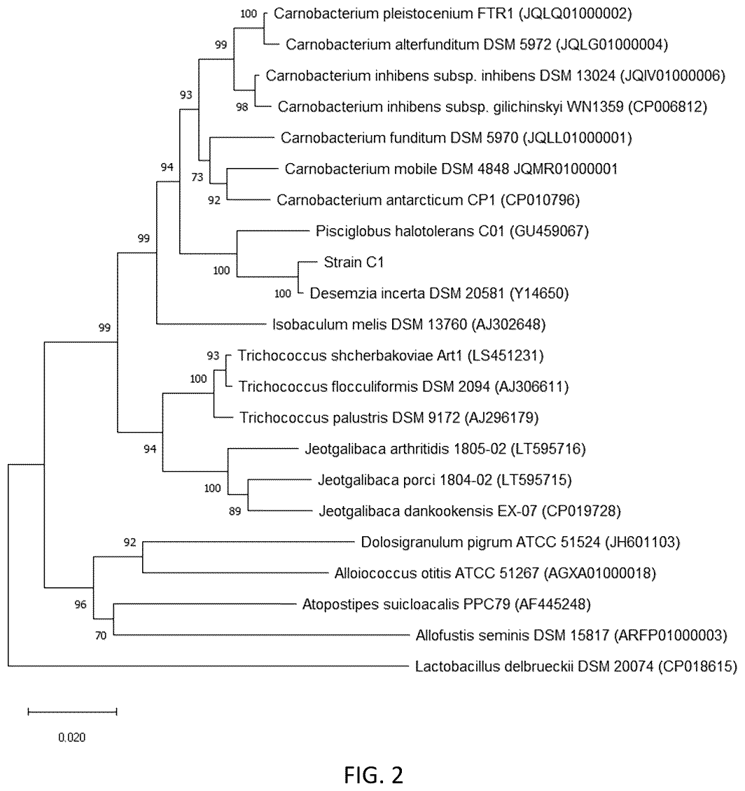

As a result of creating a phylogenetic tree based on the 16S rDNA nucleotide sequence, it was confirmed that Desemzia sp. strain C1 belonged to the same clade as Desemzia inserta and was clearly separated from other genera. Desemzia sp. strain C1 was closely related to Desemzia inserta in the phylogenetic tree, but Desemzia sp. strain C1 and Desemzia inserta belonged to different phylogenetic branches so that they could not be considered the same species. Therefore, Desemzia sp. strain C1 was identified to belong to the genus Desemzia, but was not classified as a specific species (FIG. 2).

The genus Desemzia is listed in Bergey's manual as Gram-positive, microaerophilic, fermenting bacteria. Since isolation and culture of the genus Desemzia from the ovary of cicada was first reported by Steinhaus in 1941, cases of human and mammalian infection with the genus Desemzia have not been reported, and thus the genus Desemzia is considered nonpathogenic bacteria.

### [Example 3] Growth and Hydrogen Peroxide (H₂O₂) Production of Desemzia Sp. Strain C1

Growth and hydrogen peroxide (H₂O₂) production of Desemzia sp. strain C1 were examined using, as a comparative strain, Sterptococcus oralis supsp. oralis strain (KACC 13048^{T}) known to produce hydrogen peroxide (H₂O₂). The growth of the microorganism was analyzed at a wavelength of 600 nm using a spectrophotometer, and hydrogen peroxide (H₂O₂) produced by the microorganism was quantified using Amplex™ red hydrogen peroxide/peroxidase assay kit.

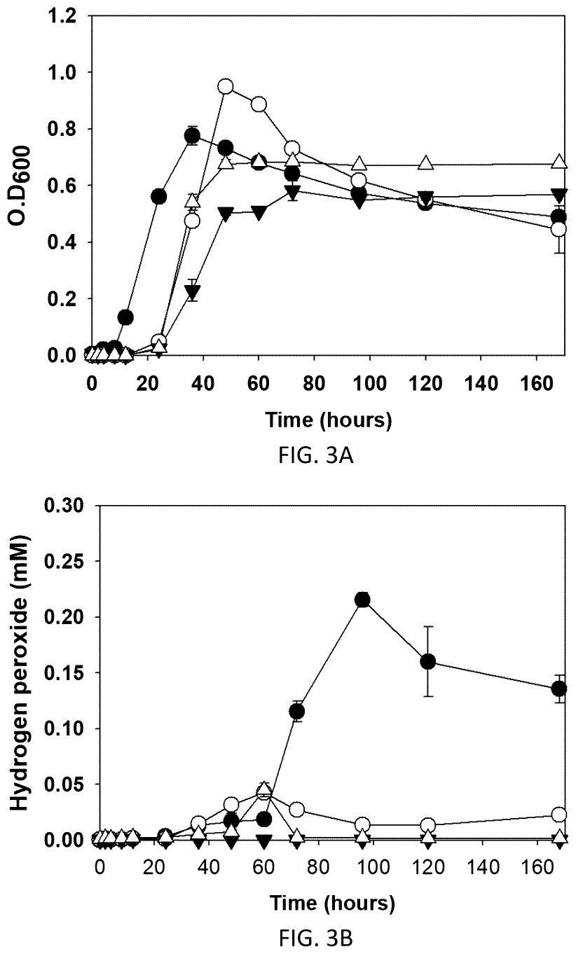

The bacteria were grown in brain heart infusion (BHI) broth and Trypticase soy yeast extract (TSBY) broth, and bacterial growth (O.D.₆₀₀) and hydrogen peroxide (H₂O₂) production were measured with time. It was confirmed that Desemzia sp. strain C1 grew better than S. oralis in both BHI and TSBY broths, and Desemzia sp. strain C1 produced a maximum of 0.23 mM hydrogen peroxide (H₂O₂) in BHI, which was 4.6 times higher than that of S. oralis (0.05 mM) (FIGS. 3A and 3B).

### [Example 4] Hydrogen Peroxide (H₂O₂) Production by Desemzia Sp. Strain C1 Depending on Substrate

In order to examine the amount of hydrogen peroxide (H₂O₂) produced by Desemzia sp. strain C1 depending on a substrate of a hydrogen peroxide (H₂O₂)-producing enzyme, a resting-cell (O.D.₆₀₀=2.5) assay was performed. 10 mM of each of glucose, lactate, pyruvate and oxalate, which are substrates of the widely known hydrogen peroxide (H₂O₂)-producing enzymes glucose oxidase, lactate oxidase, pyruvate oxidase and oxalate oxidase, was added to minimal mineral medium, and no substrate was added to a control group.

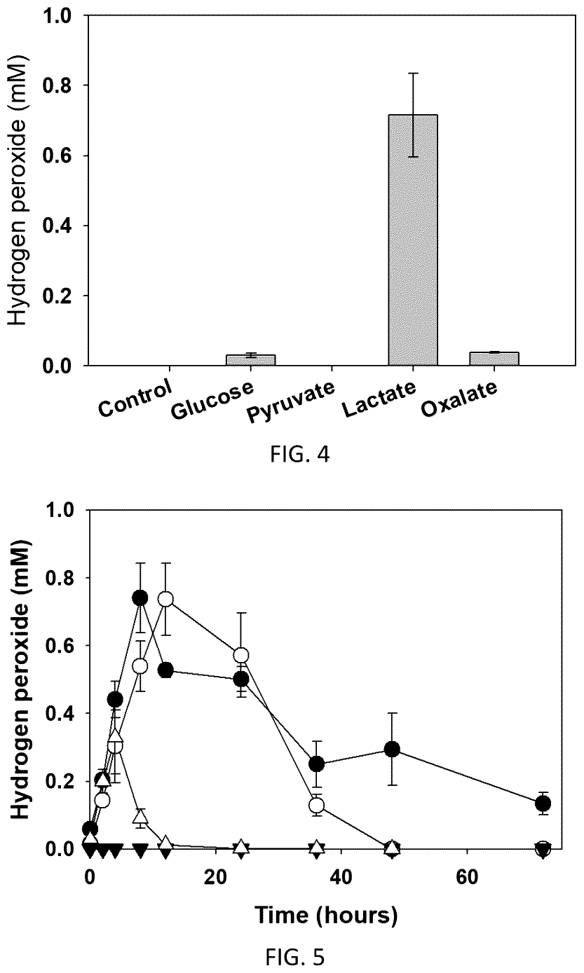

When Desemzia sp. strain C1 was cultured for 8 hours using 10 mM lactate as a substrate, it produced 0.75 mM hydrogen peroxide (H₂O₂). Meanwhile, when oxalate or glucose was used as a substrate, Desemzia sp. strain C1 produced 0.05 mM hydrogen peroxide (H₂O₂). In addition, in a group in which pyruvate was used as a substrate and in the control group, no hydrogen peroxide (H₂O₂) production was observed (FIG. 4).

### [Example 5] Hydrogen Peroxide (H₂O₂) Production by Desemzia Sp. Strain C1 Using Lactate

It was predicted that lactate would be required for the hydrogen peroxide (H₂O₂) production mechanism of Desemzia sp. strain C1. Desemzia sp. strain C1 was inoculated into a minimal mineral medium containing 10 mM lactate as a substrate so that the cell concentration (O.D.₆₀₀) reached 0.5, 1, 2.5 or 5. Periodic sampling was performed and hydrogen peroxide (H₂O₂) produced by the microorganism was quantified. The maximum production of hydrogen peroxide (H₂O₂) by Desemzia sp. strain C1 was similar (about 0.75 mM) between cell concentrations at the initial stage, but when O.D.₆₀₀ was 1, the highest concentration of hydrogen peroxide (H₂O₂) was produced after 8 hours of culture, which was the shortest time. However, when O.D.₆₀₀ was 5, the maximum production of hydrogen peroxide (H₂O₂) after 12 hours of culture was 0.3 mM, which was significantly lower than those at the other cell concentrations (FIG. 5).

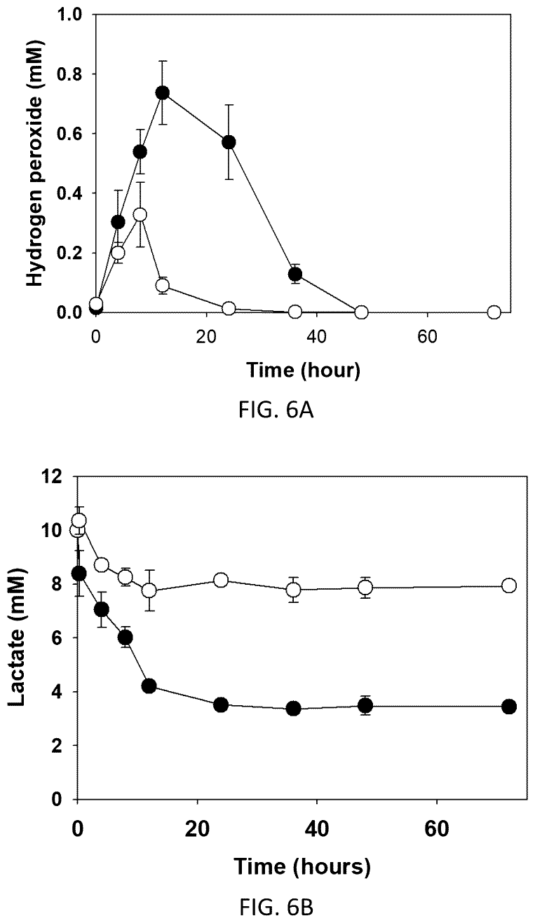

As a result of comparing hydrogen peroxide (H₂O₂) production between Desemzia sp. strain C1 and S. oralis under the condition where 10 mM lactate was used as a substrate at a cell concentration (O.D.₆₀₀) of 2.5, it was confirmed that Desemzia sp. strain C1 produced a maximum of 0.75 mM hydrogen peroxide (H₂O₂) after 8 hours of culture, and S. oralis produced a maximum of 0.25 mM hydrogen peroxide (H₂O₂) after 6 hours of culture (FIGS. 6A and 6B). Desemzia sp. strain C1 and S. oralis did not consume any more lactate after using 6 mM and 2 mM lactate, respectively, after 12 hours of culture.

From the above results, it was confirmed that, in the resting-cell assay using 10 mM lactate, the method of producing hydrogen peroxide (H₂O₂) using Desemzia sp. strain C1 induced about 3.4 times higher hydrogen peroxide (H₂O₂) production than the method of producing hydrogen peroxide (H₂O₂) using a nutrient broth such as BHI or TSBY.

Although the present disclosure has been described in detail with reference to the specific features, it will be apparent to those skilled in the art that this description is only of a preferred embodiment thereof, and does not limit the scope of the present disclosure. Thus, the substantial scope of the present disclosure will be defined by the appended claims and equivalents thereto.

Desemzia sp. strain C1 was deposited on Nov. 21, 2022 with the depository of the Korea Research Institute of Bioscience and Biotechnology, Accession number: KCTC15203BP

What is claimed is:

1. A method of producing hydrogen peroxide (H₂O₂) using a microorganism of the genus Desemzia.

2. The method according to claim 1, wherein the microorganism is a microorganism having a 16S rDNA represented by SEQ ID NO: 1.

3. The method according to claim 1, wherein the microorganism is a microorganism comprising a nucleic acid represented by SEQ ID NO: 1.

4. The method according to claim 1, wherein the microorganism uses lactate oxidase.

5. The method according to claim 1, wherein the microorganism is nonpathogenic.

6. The method according to claim 1, wherein the microorganism belongs to the same clade as Desemzia inserta.

7. The method according to claim 1, wherein the microorganism belongs to a phylogenetic branch distinct from Desemzia inserta.

8. The method according to claim 1, wherein the microorganism is a Desemzia sp. strain C1 (accession number KCTC15203BP).

9. A composition for degrading pollutants comprising a hydrogen peroxide produced by the method according to claim 1.

10. The composition according to claim 9, wherein the pollutants are organic pollutants.

11. A composition for degrading pollutants comprising a hydrogen peroxide produced by a microorganism comprising a nucleic acid represented by SEQ ID NO: 1.

12. The composition according to claim 13, wherein the microorganism uses lactate oxidase.

13. A composition for producing hydrogen peroxide comprising a microorganism of the genus Desemzia.

14. A composition for producing hydrogen peroxide comprising a 20 microorganism comprising a nucleic acid represented by SEQ ID NO: 1.

15. The composition according to claim 14, wherein the microorganism is a Desemzia sp. strain C1 (accession number KCTC15203BP).

16. A method for degrading one or more pollutants, the method comprising producing hydrogen peroxide by a method according to claim 1, and contacting the one or more pollutants with the hydrogen peroxide under conditions sufficient to degrade the pollutant.

17. The method according to claim 16, wherein the microorganism is a microorganism having a 16S rDNA represented by SEQ ID NO: 1.


SEQUENCE LISTING (1 sequence)

SEQ ID NO: 1 - DNA, 1408 bp, Desemzia sp.
  source 1..1408: mol_type = genomic DNA
cgcttcctaccggtgcttgcaccgggaaagaagagtggcggacgggtgagtaacacgtgggtaacctacctacaagcgggggataacattcggaaacggatgctaataccgcatagatctttttgtctcctggcagaaagaagaaagacggtttcggctgtcacttgtagatggacccgcggcgcattagttagttggtgaggtaatggctcaccaagacgatgatgcgtagccgacctgagagggtgatcggccacactgggactgagacacggcccagactcctacgggaggcagcagtagggaatcttccgcaatggacgaaagtctgacggagcaacgccgcgtgagtgaagaaggttttcggatcgtaaaactctgttgttagagaagaacaaggatgagagtaactgctcgtcccttgacggtatctaaccagaaagccatggctaactacgtgccagcagccgcggtaatacgtagatggcaagcgttgtccggatttattgggcgtaaagcgagcgcaggcggttctttaagtctgatgtgaaagcccctggctcaaccaggggagggtcattggaaactggagaacttgagtgcagaagaagaaagtggaattccatgtggtagcggtgaaatgcgtagatatatggaggaacaccagtggcgaaggcgactttctggtctgtaactgacgctgaggctcgaaagcatggggagcaaacaggattagataccttggtagtccatgccgtaaacgatgagtgttaagtgtgggagggtttccgcctttcagtgctgcagttaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattattagttgccagcattcagttgggcactctagtgagactgccggtgataaaccggaggaaggtggggatgacgtcaaatcatcatgccccttatgacctgggctacacacgtgctacaatggatggtacaacgagtcgcaaaaccgcgaggttaagctaatctcttaaagccattctcagttcggattgtaggctgcaattcgcctacatgaagccggaatcgctagtaatcgcggatcagaacgccgcggtgaatacgttcccgggccttgtacacaccgcccgtcacaccacgagagtttgtaacacccaagtcggtgaggtaaccttcgggagccagccg